(19) United States
(12) Patent Application Publication
(10) Pub. No.: US 2006/0085009 A1
(43) Pub. Date: Apr. 20, 2006

(54) Implants and methods for treating bone

(76) Inventors: Csaba Truckai, Saratoga, CA (US); John H. Shadduck, (US)

Correspondence Address: John H. Shadduck, 350 Sharon Park Drive #B23, Menlo Park, CA, 94025, US

(21) Appl. No.: 11/199,582

(22) Filed: Aug. 8, 2005

Related U.S. Application Data
(60) Provisional application No. 60/600,012, filed on Aug. 9, 2004.

(51) Int. Cl. A61F 2/34

(52) U.S. Cl. 606/94

(57) ABSTRACT

This invention relates to biomedical implants for filling, supporting or treating bone. In one embodiment, the implant comprises an electrospun polymer scaffold that is thereafter plated with a metal to provide a selected high modulus. Such an implant can be fabricated with a selected porosity for tissue ingrowth. The implant can be further provided with a varied modulus along the length of the implant body for inducing bending of the implant for packing in a bone. In another embodiment, the implant is fabricated in an elongated configuration for introducing into bone to treat a vertebral fracture. In another embodiment, the implant can be configured with helical threads for helically driving the implant into a bone.

### CROSS-REFERENCE TO RELATED APPLICATIONS

This application claims benefit of Provisional U.S. Patent Application Ser. No. 60/600,012 filed Aug. 9, 2004 titled Orthopedic Scaffold Implants, Methods of Use and Methods of Fabrication. This application also is related to the following Provisional U.S. Patent Applications: Ser. No. 60/590,588 filed Jul. 16, 2004 titled Orthopedic Scaffold Implants, Methods of Use and Methods of Fabrication; Ser. No. 60/590,597 filed Jul. 23, 2004 titled Orthopedic Scaffold Implants, Methods of Use and Methods of Fabrication; and Ser. No. 60/590,598 filed Jul. 23, 2004 titled Orthopedic Scaffold Implants, Methods of Use and Methods of Fabrication. The entire contents of all of the above cross-referenced applications are hereby incorporated by reference in their entirety and should be considered a part of this specification.

### BACKGROUND OF THE INVENTION

1. Field of the Invention

This invention relates to filament-like implants for filling and supporting osteoporotic bones. The filament-like implants have varied bending strength along an axis of the filament to provide deformable axial regions together non-deformable axial regions. The filament-like implants can be introduced with or without a bone cement such as PMMA.

2. Description of the Related Art

Osteoporotic fractures are prevalent in the elderly, with an annual estimate of 1.5 million fractures in the United States alone. These include 750,000 vertebral compression fractures (VCFs) and 250,000 hip fractures. The annual cost of osteoporotic fractures in the United States has been estimated at $13.8 billion. The prevalence of VCFs in women age 50 and older has been estimated at 26%. The prevalence increases with age, reaching 40% among 80-year-old women. Medical advances aimed at slowing or arresting bone loss from aging have not provided solutions to this problem. Further, the affected population will grow steadily as life expectancy increases. Osteoporosis affects the entire skeleton but most commonly causes fractures in the spine and hip. Spinal or vertebral fractures also have serious consequences, with patients suffering from loss of height, deformity and persistent pain which can significantly impair mobility and quality of life. Fracture pain usually lasts 4 to 6 weeks, with intense pain at the fracture site. Chronic pain often occurs when one level is greatly collapsed or multiple levels are collapsed.

Postmenopausal women are predisposed to fractures, such as in the vertebrae, due to a decrease in bone mineral density that accompanies postmenopausal osteoporosis. Osteoporosis is a pathologic state that literally means “porous bones”. Skeletal bones are made up of a thick cortical shell and a strong inner meshwork, or cancellous bone, of collagen, calcium salts and other minerals. Cancellous bone is similar to a honeycomb, with blood vessels and bone marrow in the spaces. Osteoporosis describes a condition of decreased bone mass that leads to fragile bones which are at an increased risk for fractures. In an osteoporotic bone, the sponge-like cancellous bone has pores or voids that increase in dimension, making the bone very fragile. In young, healthy bone tissue, bone breakdown occurs continually as the result of osteoclast activity, but the breakdown is balanced by new bone formation by osteoblasts. In an elderly patient, bone resorption can surpass bone formation thus resulting in deterioration of bone density. Osteoporosis occurs largely without symptoms until a fracture occurs.

Vertebroplasty and kyphoplasty are recently developed techniques for treating vertebral compression fractures. Percutaneous vertebroplasty was first reported by a French group in 1987 for the treatment of painful hemangiomas. In the 1990's, percutaneous vertebroplasty was extended to indications including osteoporotic vertebral compression fractures, traumatic compression fractures, and painful vertebral metastasis. In one percutaneous vertebroplasty technique, bone cement such as PMMA (polymethylmethacrylate) is percutaneously injected into a fractured vertebral body via a trocar and cannula system. The targeted vertebrae are identified under fluoroscopy. A needle is introduced into the vertebral body under fluoroscopic control to allow direct visualization. A transpedicular (through the pedicle of the vertebrae) approach is typically bilateral but can be done unilaterally. The bilateral transpedicular approach is typically used because inadequate PMMA infill is achieved with a unilateral approach.

In a bilateral approach, approximately 1 to 4 ml of PMMA are injected on each side of the vertebra. Since the PMMA needs to be forced into cancellous bone, the technique requires high pressures and fairly low viscosity cement. Since the cortical bone of the targeted vertebra may have a recent fracture, there is the potential of PMMA leakage. The PMMA cement contains radiopaque materials so that when injected under live fluoroscopy, cement localization and leakage can be observed. The visualization of PMMA injection and extravasion are critical to the technique and the physician terminates PMMA injection when leakage is evident. The cement is injected using small syringe-like injectors to allow the physician to manually control the injection pressures.

Kyphoplasty is a modification of percutaneous vertebroplasty. Kyphoplasty involves a preliminary step that comprises the percutaneous placement of an inflatable balloon tamp in the vertebral body. Inflation of the balloon creates a cavity in the bone prior to cement injection. Further, the proponents of percutaneous kyphoplasty have suggested that high pressure balloon-tamp inflation can at least partially restore vertebral body height. In kyphoplasty, it has been proposed that PMMA can be injected at lower pressures into the collapsed vertebra since a cavity exists to receive the cement—which is not the case in conventional vertebroplasty.

The principal indications for any form of vertebroplasty are osteoporotic vertebral collapse with debilitating pain. Radiography and computed tomography must be performed in the days preceding treatment to determine the extent of vertebral collapse, the presence of epidural or foraminal stenosis caused by bone fragment retropulsion, the presence of cortical destruction or fracture and the visibility and degree of involvement of the pedicles. Leakage of PMMA during vertebroplasty can result in very serious complications including compression of adjacent structures that necessitate emergency decompressive surgery.

Leakage or extravasion of PMMA is a critical issue and can be divided into paravertebral leakage, venous infiltration, epidural leakage and intradiscal leakage. The exothermic reaction of PMMA carries potential catastrophic consequences if thermal damage were to extend to the dural sac, cord, and nerve roots. Surgical evacuation of leaked cement in the spinal canal has been reported. It has been found that leakage of PMMA is related to various clinical factors such as the vertebral compression pattern, and the extent of the cortical fracture, bone mineral density, the interval from injury to operation, the amount of PMMA injected and the location of the injector tip. In one recent study, close to 50% of vertebroplasty cases resulted in leakage of PMMA from the vertebral bodies. See Hyun-Woo Do et al, “The Analysis of Polymethylmethacrylate Leakage after Vertebroplasty for Vertebral Body Compression Fractures”, Jour. of Korean Neurosurg. Soc. Vol. 35, No. 5 (May 2004) pp. 478-82, (http://www.j.kns.or.kr/htm/abstract.asp?no=0042004086).

Another recent study was directed to the incidence of new VCFs adjacent to the vertebral bodies that were initially treated. Vertebroplasty patients often return with new pain caused by a new vertebral body fracture. Leakage of cement into an adjacent disc space during vertebroplasty increases the risk of a new fracture of adjacent vertebral bodies. See Am. J. Neuroradiol. 2004 February; 25(2): 175-80. The study found that 58% of vertebral bodies adjacent to a disc with cement leakage fractured during the follow-up period compared with 12% of vertebral bodies adjacent to a disc without cement leakage.

Another life-threatening complication of vertebroplasty is pulmonary embolism. See Bernhard, J. et al., “Asymptomatic diffuse pulmonary embolism caused by acrylic cement: an unusual complication of percutaneous vertebroplasty”, Ann. Rheum. Dis. 2003; 62:85-86. The vapors from PMMA preparation and injection are also cause for concern. See Kirby, B., et al., “Acute bronchospasm due to exposure to polymethylmethacrylate vapors during percutaneous vertebroplasty”, Am. J. Roentgenol. 2003; 180:543-544.

Another disadvantage of PMMA is its inability to undergo remodeling—and the inability to use the PMMA to deliver osteoinductive agents, growth factors, chemotherapeutic agents and the like. Yet another disadvantage of PMMA is the need to add radiopaque agents which lower its viscosity with unclear consequences on its long-term endurance.

In both higher pressure cement injection (vertebroplasty) and balloon-tamped cementing procedures (kyphoplasty), the methods do not provide for well controlled augmentation of vertebral body height. The direct injection of bone cement simply follows the path of least resistance within the fractured bone. The expansion of a balloon also applies compacting forces along lines of least resistance in the collapsed cancellous bone. Thus, the reduction of a vertebral compression fracture is not optimized or controlled in high pressure balloons as forces of balloon expansion occur in multiple directions.

In a kyphoplasty procedure, the physician often uses very high pressures (e.g., up to 200 or 300 psi) to inflate the balloon which first crushes and compacts cancellous bone. Expansion of the balloon under high pressures close to cortical bone can fracture the cortical bone, or cause regional damage to the cortical bone that can result in cortical bone necrosis. Such cortical bone damage is highly undesirable and results in weakened cortical endplates.

Kyphoplasty also does not provide a distraction mechanism capable of 100% vertebral height restoration. Further, the kyphoplasty balloons under very high pressure typically apply forces to vertebral endplates within a central region of the cortical bone that may be weak, rather than distributing forces over the endplate.

There is a general need to provide systems and methods for use in treatment of vertebral compression fractures that provide a greater degree of control over introduction of bone support material, and that provide better outcomes. Embodiments of the present invention meet one or more of the above needs, or other needs, and provide several other advantages in a novel and non-obvious manner.

### SUMMARY OF THE INVENTION

The invention provides a implants and methods for the prophylactic treatment of osteoporotic bone or for treating a vertebra that has a compression fracture. The invention is also useful in correcting and supporting bones in other abnormalities such as bone tumors and cysts, avascular necrosis of the femoral head and tibial plateau fractures. In an exemplary embodiment, the bone abnormality can be accessed in a minimally invasive manner and the implants can be directly introduced into cancellous bone.

In one embodiment, the implant comprises an electrospun polymer volume that functions as a porous scaffold which is thereafter plated with a metal to provide a selected high modulus. Such an implant can be fabricated with a selected porosity for tissue ingrowth. The implant can be further provided with a varied modulus along the length of the implant body for inducing bending of the implant for packing in a bone. In another aspect of the invention, an implant can be fabricated in an elongated configuration for axially pushing through an introducer into bone wherein the implant deforms into a convoluted mass in the bone. In another aspect of the invention, the implant can be configured with helical threads for helically driving the implant into a bone.

In another aspect of the invention, an introducer sleeve is configured with anchoring means comprising threads for engaging bone. The threads grip the bone to prevent any possible outward migration when injecting implants and/or bone cement into a bone.

In another aspect of the invention, an elongate implant can be variably heat treated for providing the implant with alternating rigid and bendable or fracturable regions for creating a bone support volume. These and other aspects of the present invention will become readily apparent upon further review of the following drawings and specification.

### BRIEF DESCRIPTION OF THE DRAWINGS

In order to better understand the invention and to see how it may be carried out in practice, some preferred embodiments are next described, by way of non-limiting examples only, with reference to the accompanying drawings, in which like reference characters denote corresponding features consistently throughout similar embodiments in the attached drawings.

FIG. 1 is a side view of a segment of a spine with a vertebra having a compression fracture that can be repaired with the present invention, showing an introducer in a method of the invention.

FIG. 2 is a cross-sectional view of the vertebra and abnormality of FIG. 1 with spaces created therein by compaction, balloon expansion, reaming or other means.

FIG. 3 is a micrograph of a non-woven matrix of polymer fibers created by an electrospinning process.

FIG. 4 is block diagram describing the steps in a method the invention for fabricating biomedical implants or scaffolds of the invention.

FIG. 5 is a schematic view of a skeletal sacrificial form of the invention used for fabricating an exemplary scaffold.

FIG. 6 is a schematic view of a completed scaffold of the invention that is formed over the skeletal sacrificial form of FIG. 5.

FIG. 7 is a greatly enlarged schematic view of an electrospun polymer matrix that is formed over the skeletal sacrificial form of FIG. 5.

FIG. 8 is a greatly enlarged schematic view similar to FIG. 7 that illustrates layers of metal plating over the electrospun matrix of FIG. 7.

FIG. 9 is a schematic view similar to FIG. 2 that illustrates a deformable filament having non-deformable axial regions with intermediate deformable or fracturable axial regions and an introducer in phantom view.

FIG. 10 is block diagram describing the steps of a method the invention for fabricating a variable modulus implant for treating osteoporotic bone.

FIG. 11 is a sectional view of a vertebra infilled with at least one implant as in FIG. 9 with alternating non-deformable axial regions and bendable axial regions.

FIG. 12A is a side view of the distal portion of an introducer showing anchoring means in the form of helical threads for engaging bone.

FIG. 12B is a view of the distal portion of an introducer showing alternative anchoring means in the form of an outwardly flexing surface.

FIG. 12C is a view of an introducer showing alternative anchoring means in the form of at least one barb element.

FIG. 12D is a view of an introducer showing alternative anchoring means in the form of an expandable balloon.

FIG. 13 is a schematic view similar to FIG. 9 that illustrates an implant having alternating non-deformable and deformable axial regions, wherein the implant is threaded for helical introduction into bone from the at least partly threaded bore of an introducer.

FIG. 14A is a schematic view of an electrospun implant that has at least one flexible to rigid transition.

FIG. 14B is a sectional view of another electrospun implant in an artificial disc configuration that transitions from rigid to flexible to rigid.

### DETAILED DESCRIPTION OF THE INVENTION

The present invention relates to implants and methods for treating bone abnormalities such as a vertebral fracture. More specifically, the invention is directed to a porous biocompatible metal or composite scaffold that has pores of a selected dimension, including scaffold ligaments that can have nanoscale pores. The implant can further have a very high modulus or a gradient in modulus. The novel material is suited for several orthopedic applications including treatment of compression fractures, spine fusion and joint replacement procedures.

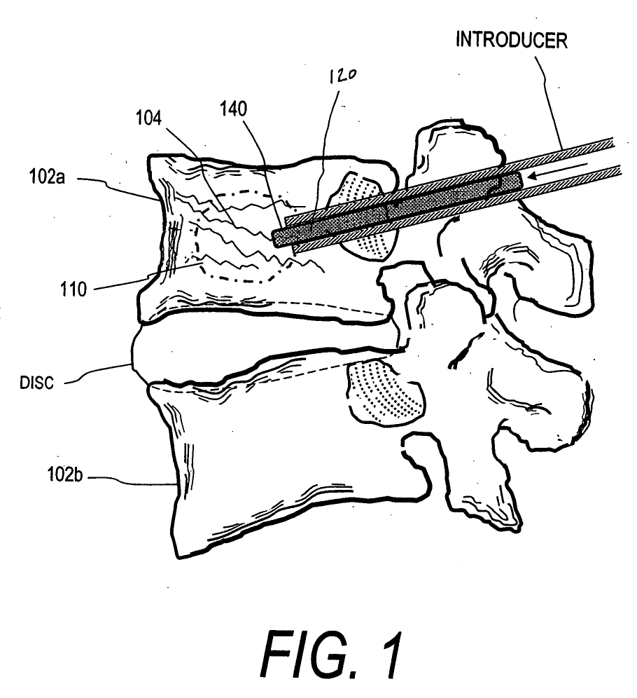

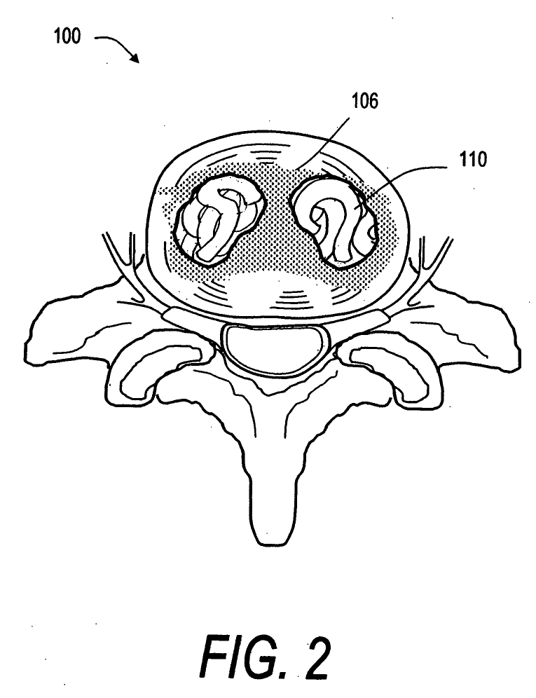

FIG. 1 shows a spine segment 100 in a patient with an osteoporotic vertebra 102a that is susceptible to a compression fracture 104. The cancellous bone in the interior of the vertebra can fracture and collapse. The targeted treatment site is indicated at 110. The targeted site or sites 110 as indicated in FIG. 2 can be supported by direct injection of at least one deformable filament 120 corresponding to the invention under suitable high injection pressure. Alternatively, a cavity can be created to receive filaments 120 with the cavity created by the cutting of cancellous bone or the compaction of cancellous bone by a balloon or other compaction means such as an ultrasound or low frequency mechanical vibration device. In one embodiment, the cancellous bone is cut or reamed and extracted from the vertebra. The space 110 can comprise a single space or a multiplicity of spaces.

In general, the system and method of invention relates to minimally invasive percutaneous interventions for providing bone support with an implant scaffold, for example in treating compression a fractures in a vertebra. A targeted treatment region 110 (FIGS. 1 and 2) in a vertebra can be accessed through a minimally invasive percutaneous access path, for example less than about 5 mm. in diameter. The introducer of the invention preferably is less than about 3 to 4 mm.

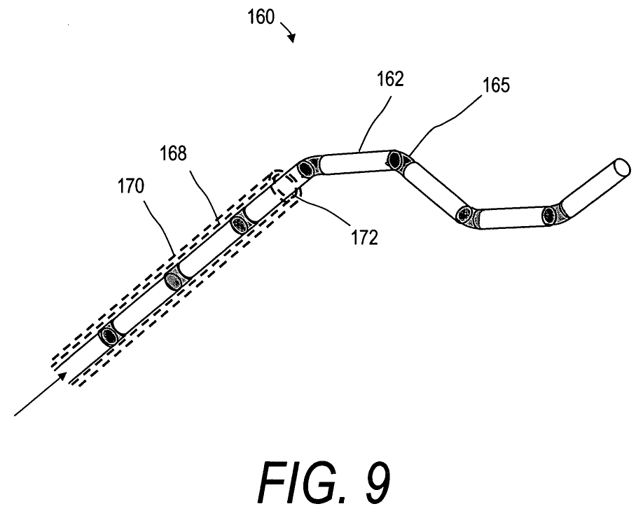

In a preferred embodiment, as will be described below in FIG. 9, the deformable filaments 120 are open cell scaffolds that have a variable elastic modulus with first axial portions having a modulus of 1 to 100 GPa to provide crush resistance with second intermediate axial regions being bendable with modulus of less than 1.0 GPa.

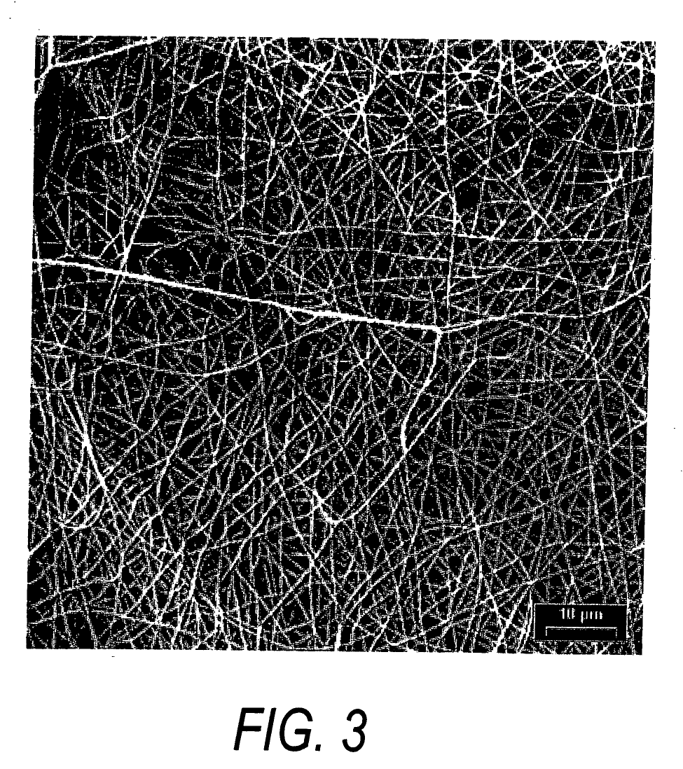

Now turning to FIGS. 3-8, a method of making the scaffold is shown. FIG. 3 is a view of greatly enlarged, very fine electrospun polymer fibers. Electrospinning is a fiber spinning method that has been used for a decade or more to produce extremely fine fibers ranging form micron to submicron diameters. The electrospinning process requires charging a polymer solution or melt with thousands of volts. The “spinning” occurs when the polymer solution is charged with 5 kV to 30 kV which is sufficient to overcome surface tension forces of the polymer, and wherein a free surface of charged polymer will produce fine jets of the liquid solution from a nozzle. The jetted solution is drawn under high velocity toward a grounded target. The jet splits a number of times near the liquid surface. Before the jetted polymer solution reaches the grounded target, it typically is drawn in a series of loops as the solution solidifies into a fiber. The electrospinning process results in an interconnected web of filaments on the surface of a grounded target. In this disclosure, the electrospun material is alternately described as a scaffold or matrix.

Of particular interest, the electrospun scaffolds have can be designed to have very high surface areas, for example up to 10 m² per gram. Further, the scaffolds can have a void or open volume that is very high, with a mean pore cross section that can be within a wide range of selected micron and submicron dimensions. Fiber sizes in the range of 10 nm and smaller are reported in research projects, and fibers in the 100 nm to 500 micron diameter range are easily spun. Fiber diameter, among other things, depends upon solution viscosity, field strength, field uniformity and jetting pressure.

In the prior art, several disclosures relate to electrospun collagen and polymers for soft tissue fillers, organ walls and the like. For example, U.S. Pat. No. 4,323,525 described a process for fabricating tubular vessel by electrospinning a polymer that forms into fibers material. The method draws spun fiber to charged tubular collector which rotates about an axis to form the tubular material. In U.S. Pat. No. 4,689,186, the author disclosed another process for fabricating tubular products by electrostatically spinning the fiber-forming liquid that included a polyurethane. The disclosure describes additional electrodes that facilitate fiber spinning and deposition. In published U.S. Patent Application 20020090725, Simpson et al. disclose processes for electrospinning collagen to form an extracellular matrix that can be used for tissue engineering purposes.

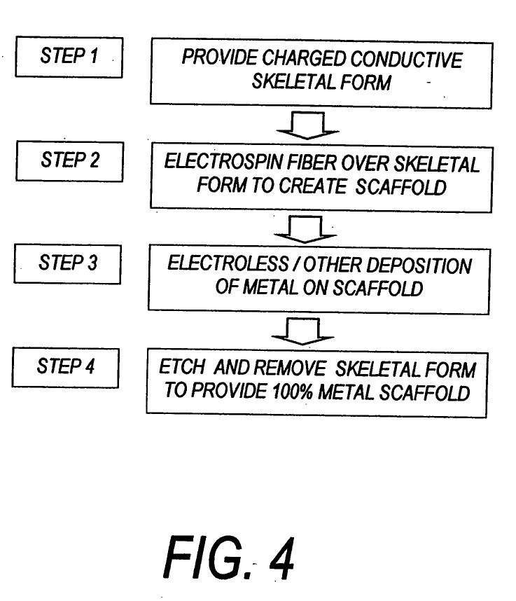

A fabrication method of the invention is described in the block diagram of FIG. 4. In general, an electrospinning method is used in conjunction with an open cell sacrificial skeletal elements 122 to rapidly create a three dimensional scaffold with soft electrospun ligaments 125 that defines a substantial open volume fraction. As can be seen in FIG. 4, the electrospun ligaments 125 thereafter are coated with a metal by electroless plating or another means wherein the metal can be deposited until a selected strength is achieved. A biomedical scaffold can be fabricated in this manner that comprises an open pore metal-surface structure in any selected shape having a predetermined microscale open pore volume, a predetermined microscale pore pattern, a predetermined microscale mean pore cross section and a predetermined microscale pore shape.

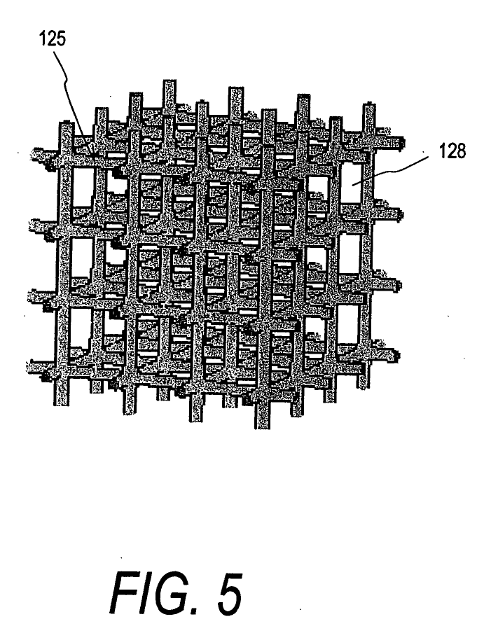

FIG. 5 illustrates an exemplary ordered skeletal form 122 that is optionally sacrificial. The axial elements 122 can be any material such as carbon fiber or metal that can serve as a ground to function as a target in the electrospinning process. While FIG. 5 illustrates the axial elements as being joined at connections of the x-axis, y-axis and z-axis, the axial elements 122 also can simply be tensioned independent elements. The form of the FIG. 5 can be configured in any shape and mounted on any type of stage to allow its rotation and/or translation during the electrospinning process.

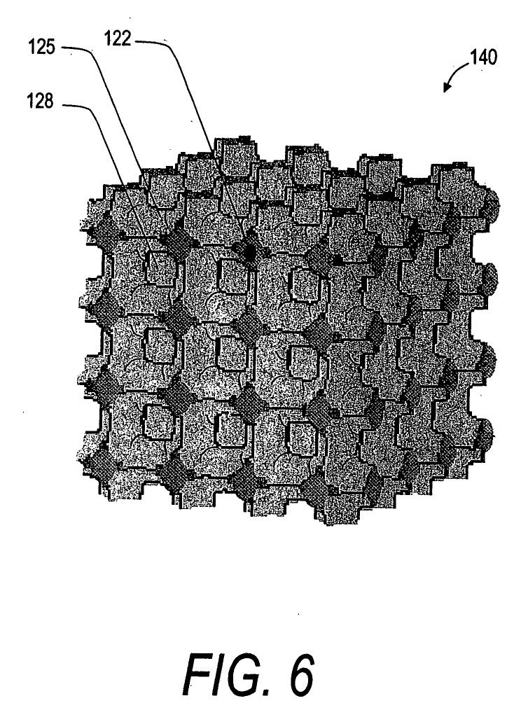

FIG. 6 illustrates scaffold 120 with struts 140 that comprises a matrix of electrospun fibers applied about the skeletal form 122 of FIG. 5 together with metal plating 144 over the entire structure to create a high modulus construct. The three dimensional scaffold filament can have a mean cross section of pores or cells 128 that is between 1 micron and 1000 microns. More preferably, the mean pore cross section is between 10 microns and 500 microns. The three dimensional scaffold as in FIG. 6 can have uniform size cells 128 or a gradient in dimension across the scaffold.

As can be understood from FIG. 6, the scaffold struts 140 will create open cells 128 that have substantially smooth surfaces which it is believed are needed for optimizing cell migration and tissue ingrowth. The pore or cell shapes can be at least one of substantially polygonal, round or oval. In another embodiment (not shown), the scaffold can be any foamed open-cell polymer that provides disordered cells. The polymer can be conductively doped to carry a charge for cooperating with electrospun fiber deposition.

Of particular interest, the scaffold ligaments 125 are also nano- or microporous which can further enhance tissue ingrowth throughout the scaffold. The three dimensional scaffold filament thus has scaffold ligaments with interconnected nanoscale pores therein. The nanoscale pores can have a mean cross section between 10 nanometers and 100 microns. More preferably, the ligaments have nanoscale interconnected void volumes having a mean cross section ranging from 50 nanometers to 50 microns.

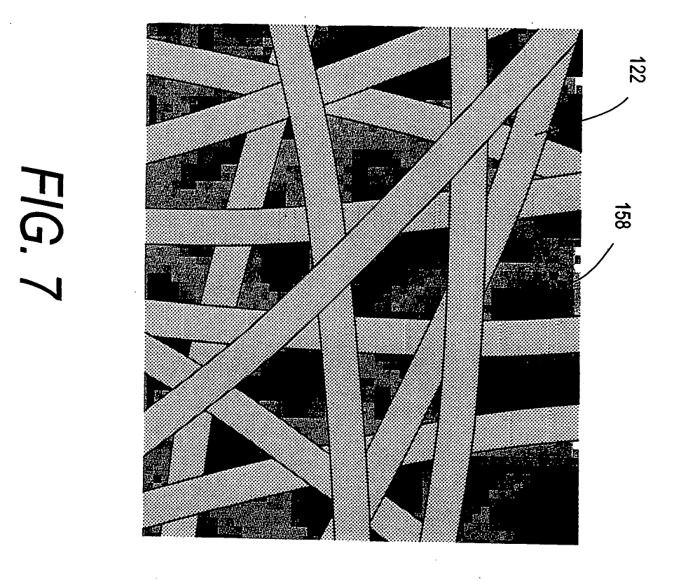

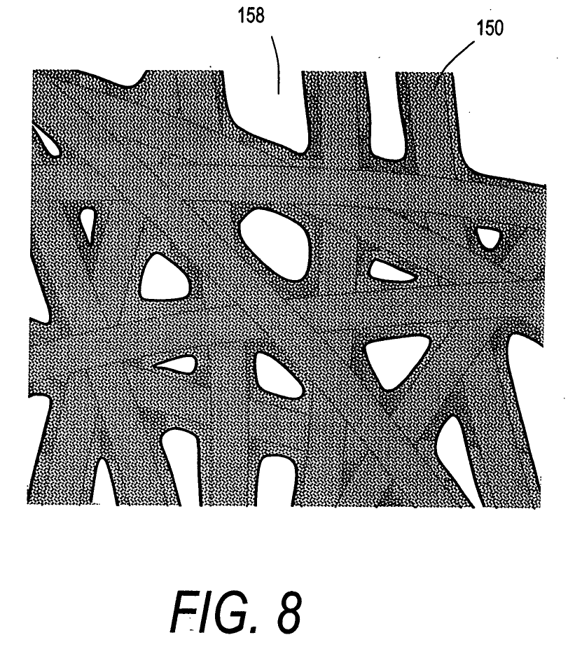

The schematic illustration of FIG. 7 illustrates a plurality of fibers in a mat or matrix as follows electrospinning. The matrix is then a soft spongy material with cotton-like characteristics. FIG. 8 is a schematic illustration of the fibers of FIG. 7 following deposition of metal layer 150 that can interconnect the fibers into ligaments and still leave nanopores 158 therein. By adding metal layers with electroless plating or other plating means, a filament scaffold can be rapidly assembled that is substantially rigid with a selected elastic modulus. A monolith of the scaffold filament can be microfabricated by the above methods to provide an elastic modulus (Young's) modulus of at least 1.0 GPa. In exemplary embodiments, the body can have a Young's modulus of at least 5.0 GPa or at least 10.0 GPa. In exemplary embodiments, as also represented in FIG. 5, the scaffold filament 120 can have an open volume fraction that ranges between 60% and 98%, and preferably ranges between 70% and 98%, and more preferably ranges between 80% and 98%.

The scope of the invention encompasses the assembly of deformable scaffold filaments as in FIGS. 2 and 9 for insertion and packing into cancellous bone in a vertebra by tamping-in at least one elongated filament. Of particular interest, each filament will be convoluted and entangled with other filaments to provide a substantially rigid monolith—thus allowing the creation of a substantially rigid porous monolith having a cross section far larger than the cross section of the bore in a conventional introducer.

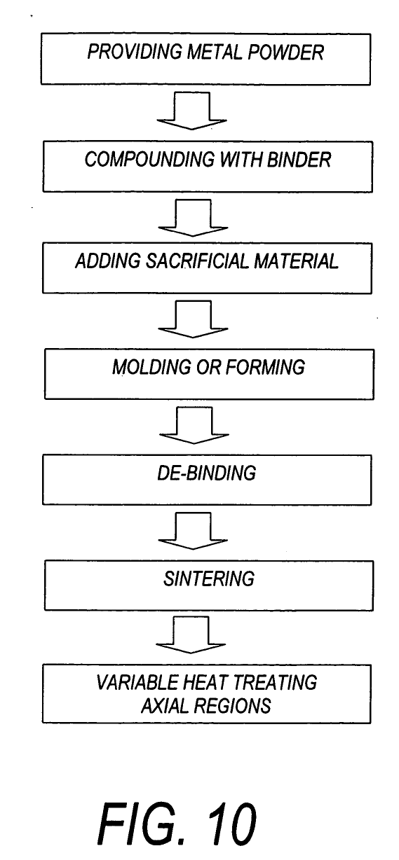

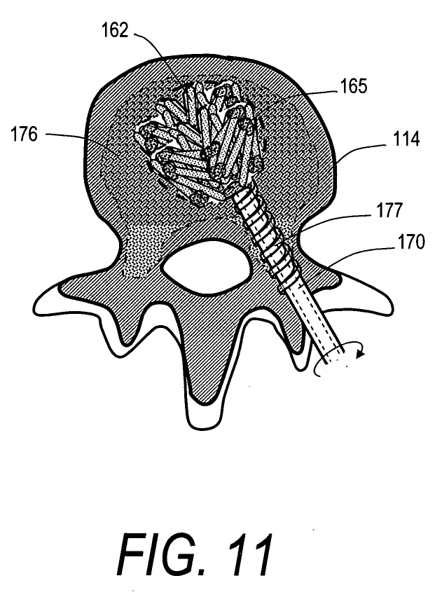

FIG. 9 illustrates an implant 160 that has axial portions 162 that are rigid and non-crushable and have a high modulus, e.g., greater than 1.0 GPa, or 5.0 GPa, or greater than 10.0 GPa. The filament 160 has intermediate axial portions 165 that are bendable and deformable with a lower modulus, e.g., less that about 1.0 GPa to allow for introduction and bending of the filaments in a vertebra as in FIG. 11. The chart of FIG. 10 describes a method of fabricating implant 160 that has alternating deformable and non-deformable axial regions, 162 and 165, respectively. Referring back to FIG. 9, the implant 160 has sufficient rigidity with sufficiently long rigid regions 162 to be pushed through a cooperating bore 168 of an introducer 170 without kinking. When exiting the introducer, the deformable regions 165 will bend or fracture to fill the targeted region of the vertebra. The bore 168 of introducer 170 can have a distal termination 172 that is oriented for axial or angular ejection of the implant, or the distal termination 172 can be angularly adjustable as provided by an adjustable end in the introducer. Various means of concentric rotatable sleeves are known in the art for providing a bore termination 172 with an adjustable exit axis.

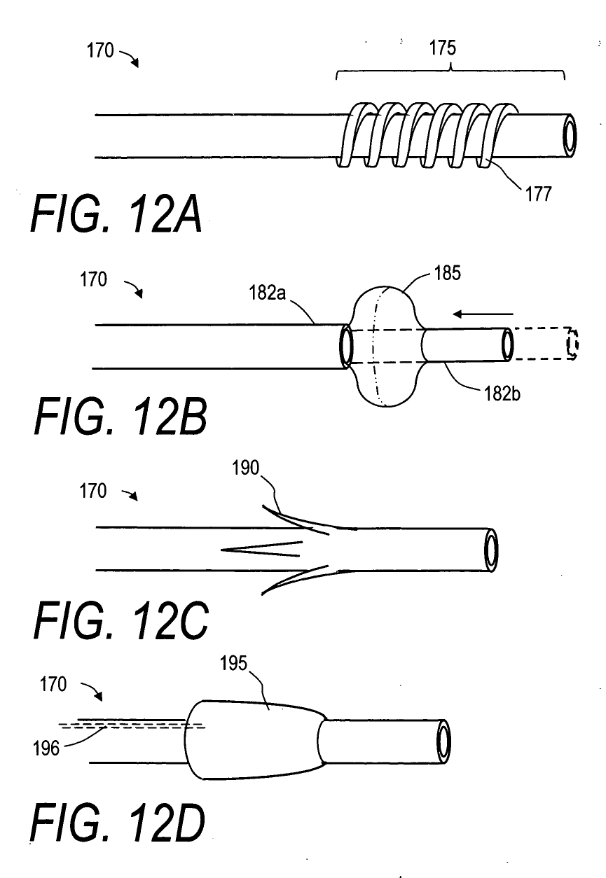

FIGS. 11 and 12A illustrate another aspect of the invention wherein the introducer 170 carries anchoring means for anchoring a distal portion 175 of the introducer in bone, for example in cancellous bone 176 of a vertebra. In the embodiment of FIGS. 11 and 12A, the introducer 170 has helical threads 177 for screwing into bone. The anchoring of the distal portion of the introducer allows for stabilization of the introducer during the injection of implants or bone cement and prevents the tendency of the injection forces to move the introducer 170 outwardly from the bone.

FIGS. 12B-12D illustrate other embodiments of the introducer 170 that have other anchoring means for engaging the distal region of introducer 170 with bone to prevent the introducer from moving proximally when very high pressures are used to inject any implant, bone fill material or bone cement. FIG. 12B illustrates a working end with first and second concentric sleeves 182a and 182b that can be used to buckle and radially expand a resilient element 185 such as a rubber member. Alternatively, the system of FIG. 12B could be configured to buckle at least one metal element as in a toggle. FIG. 12C illustrates a working end with barbs 190 that engage the bone as the structure is moved slightly proximally. Such an introducer can have a distal region that is detachable by means of a detachment mechanism, or the introducer barbs can be configured to collapse somewhat under rotation to thereby rotate and withdraw the introducer from a bone. FIG. 12D illustrates a working end with an expandable balloon structure 195 that is inflatable through lumen 196 and can be used to anchor the introducer 170 in bone.

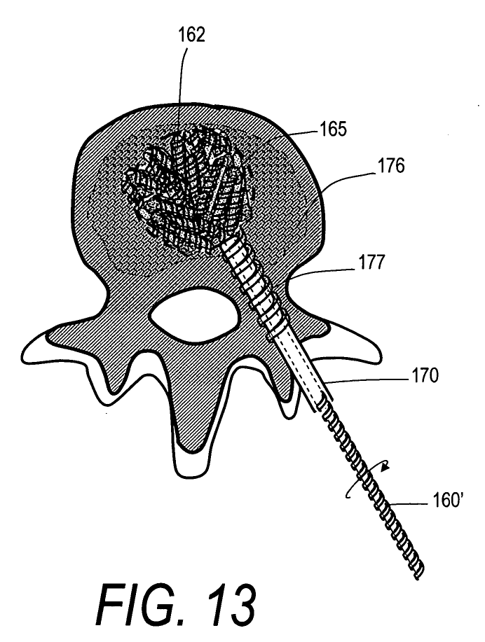

FIG. 13 illustrates another implant embodiment 160′ that is similar to that of FIG. 9 with axially-extending portions 162 that are rigid and intermediate axial portions 165 that are bendable, deformable or fracturable. The implant 160′ differs in that it is introduced into bone by helically driving the implant within bore 168 of introducer 170 that has threads that cooperate with implant threads 198 that extend about the body of implant 160′. The implant 160′ can be driven into bone with high forces with a motor drive but also can be hand driven. The deformable axial portions 165 are configured to have sufficient strength to not deform under high force application so as to insure against binding in the threaded bore 168 of the introducer. As the implant 160′ exits the introducer, any force against the implant can cause the deformable portion 165 to bend or fracture. In another embodiment, the termination 172 of the bore 168 can carry a feature that deflects the axis of the implant to fracture, bend or generally direct or re-direct the implant and it axis of introduction. In another embodiment (not shown) the implant can have a helical threads or a series of ridges to cooperate with the teeth of a gear for driving the implant axially through an introducer 170.

FIG. 10 describes a method of fabricating implant 160 or 160′ in a metal injecting molding (MIM) process to provide alternating deformable and non-deformable axial regions, 162 and 165 (see FIGS. 9, 11 and 13). Metal injection molding (MIM) is a powder metallurgy process and is a preferred method for economically fabricating the elongate implants of the invention. As background, a MIM process involves four steps, compounding (or mixing), molding, de-binding and sintering. Initially, at least one metallic powder is selected for its inherent properties, such as biocompatibility and strength as well as size and shape. Typically, the powder particles are coated with an inorganic binder. In the compounding step, the metallic powder is compounded with additional polymeric binders complemented with additives to thereby create a feedstock. The binders enable transport of the powder for molding purposes and gives the final form its rigidity in a green state. In a method of the invention, an additional “sacrificial” material component is added to the material being compounded. Thereafter, the feedstock can be injected into molds much the same as in plastic injection molding. The component that results from the molding stage is referred to as a green part. The de-binding stage of the MIM process include catalytic, thermal, solvent or water bath de-binding. The de-binding removes the bulk of the polymeric binding material. In some methods, the de-binded green part is exposed to ultraviolet light to thermoset selected other binding agents used with the metal powders. Once this has been completed, the element is sintered by being placed in a furnace at temperatures ranging from 600° F. to 2,200° F. or higher to fuse the fine powdered particles into a solid shape that retains the molded shape. The sintering step creates a monolithic body that can be further heat treating in a method of the invention to create variable modulus regions.

While MIM is a preferred method of the invention with the use of injection type molds, other similar powder metal forming processes fall within the scope of the invention. Forming methods can include cold isostatic pressing, hot isostatic pressing, rolling, extrusion and pressureless compaction.

The final step of the invention as indicated in FIG. 10 relates to variably heat treating the material to make selected axial regions have a high Young's modulus (e.g., above 5 GPa). More preferably, the higher modulus axial portions have a modulus of at least 25.0 GPa. At the same time, alternating axial regions of the element can be heat treated in a different manner to make such regions low modulus and bendable (e.g., less that 0.5 GPa). Heat treating or annealing such material to provide varied deformability can be accomplished by locally treating regions in isolation, for example with the use local heating by laser, resistive heating, etc. At the same time, local cooling can be provided if needed by cryogenic means and the like.

The invention allows for the creation of a porous bone implant for use without a liquid bone cement that polymerizes in-situ. The surfaces of the scaffold material can have a lower modulus and flex or deform to mesh in an interface with a bone surface and mesh with adjacent scaffold elements under compression. The inventive system allows creation of a porous implant without the creation of heat that occurs with bone cements. In another aspect of the invention, the vertebral body together with the implant can optionally filled with any polymer that can harden, such as a PMMA bone cement. Of particular interest, the system also may be used in a prophylactic manner with small introducers, for example, to provide bone support in vertebra of patients in advance of compression fracture. FIGS. 1 and 2 indicate that the treatment site 110 can be a single region or multiple spaced apart regions, for example accessed by two minimally invasive access pathways.

In any embodiment, the implant 120, 160 or 160′ further can be implanted with or carry any bioactive material or agent, a pharmacological agent or the like in any of the following classes: antibiotics, cortical bone material, synthetic cortical replacement material, demineralized bone material, autograft and allograft materials. The implant body also can include drugs and agents for inducing bone growth, such as bone morphogenic protein (BMP). The implants can carry the bioactive and pharmacological agents for immediate or timed release. The agent can include growth factors and the like. It should be appreciated that the implant system can be scaled to any suitable dimension and used to treating any bone infill need, whether for trauma, in conjunction with joint replacement, or for any prophylactic treatment of osteoporotic bone.

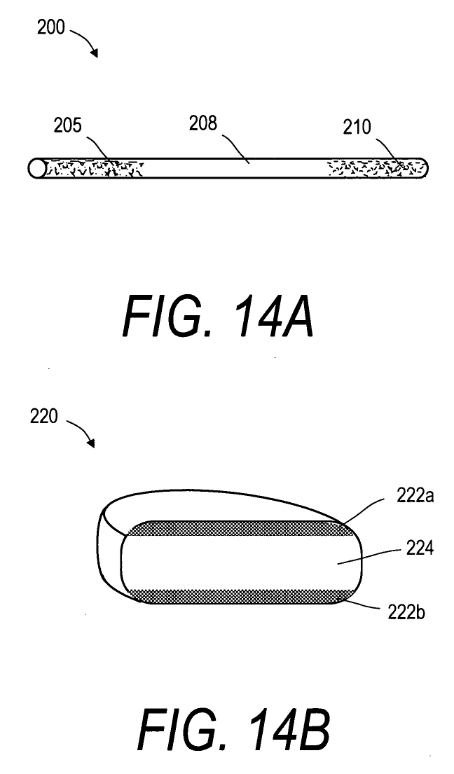

In another embodiment of the invention, an electrospun polymer structure 200 can be surface treated to provide a fabrication that transitions at a practically nanoscale level in a gradient from substantially flexible to substantially rigid. In a first end or side 205 of the implant or fabrication, the electrospun material is surface modified or coated with a polymeric material to cross-link together the electrospun fibers to provide a flexible scaffold region. The medial region 208 of the fabrication and the opposing second end or side 210 of the fabrication carries increasingly thick metallic layers (as described above) to thereby transition the implant to a rigid configuration. After the varied thickness metal plating of the medial region and second end is accomplished, the electrospun materials of the first end and the diminishing pores of the medial region can be infilled and bonded with a suitable elastomer to create a solid non-porous first end 205, if tissue ingrowth is not desired and further strength is required. In one embodiment as shown schematically in FIG. 14A, an elongated flexible-to-rigid implant 200 can be used as an artificial ligament or the like. The rigid second end 210 can be inserted and fixed in an endosteal bore which will allow for bone ingrowth therein. In another embodiment, the fabrication can transition from rigid to flexible and back to rigid or vice versa, again for use as ligaments and the like. In another embodiment, FIG. 14B illustrates a sectional view of an artificial nucleus or an artificial disc 220 that transitions from hard porous surfaces 222a and 222b allowing for bone ingrowth to a central region 224 that comprises electrospun materials with an additional infill of an elastomer to provide suitable flexibility.

The above description of the invention intended to be illustrative and not exhaustive. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

1-34. (canceled)

35. A bone implant comprising an elongate member extending along an axis, the member having alternating first non-deformable axial regions and second intermediate axial regions that are at least one of deformable, fracturable and separable.

36. A bone implant as in claim 35 wherein the elongate member is rod-like.

37. A bone implant as in claim 35 wherein the elongate member has helical threads.

38. A bone implant as in claim 35 wherein the elongate member is of at least one of a metal and a polymer.

39. A bone implant as in claim 35 wherein the metal is at least one of titanium, nickel-titanium alloy, tantalum, platinum, palladium, gold, silver, stainless steel and molybdenum.

40. A bone support implant as in claim 35 wherein the elongate member has a cross-section ranging between 0.2 mm and 5 mm.

41. A bone support implant as in claim 35 wherein the elongate member has a cross-section ranging between 1 mm and 4 mm.

42. A bone implant as in claim 35 further comprising an introducer sleeve with a bore having helical features for cooperating with the helical threads of the elongate member.

43. A method of making a bone implant, comprising the steps of:
(a) metal injection molding an elongate member that includes distributed sacrificial portions; and
(b) sacrificing the sacrificial portions thereby providing a porous monolith.

44. A method of making a bone implant as in claim 43 wherein the molding step includes placing a compounded powderized metal-polymer material in a mold.

45. A method of making a bone implant as in claim 43 wherein the sacrificing step includes at least one of solvent etching of the sacrificial portions, thermal removal of the sacrificial portions and catalytic removal of the sacrificial portions.

46. A method of making a bone implant as in claim 43 further comprising variably heat treating alternating axial regions of the elongate member thereby providing alternating non-deformable and deformable axial regions.

47. A method of making a bone implant as in claim 46 wherein the heat treating includes differentially controlling heating and cooling intervals of the alternating axial regions.

48. A method of making a bone implant as in claim 46 wherein the heat treating step includes creating axial regions having a Young's modulus of greater than 5 GPa.

49. A method of making a bone implant as in claim 46 wherein the heat treating step includes creating axial regions having a Young's modulus of less than 0.5 GPa.
